(19) United States
(12) Patent Application Publication
(10) Pub. No.: US 2009/0196832 A1
(43) Pub. Date: Aug. 6, 2009

(54) Novel edible aqueous aerosol foam

(76) Inventor: Esteban Lakos, Ciudad Jardin (AR)

Correspondence Address: SARFARAZ K. NIAZI, 20 RIVERSIDE DRIVE, DEERFIELD, IL, 60015, US

(21) Appl. No.: 12/024,052

(22) Filed: Jan. 31, 2008

(51) Int. Cl. A61K 9/12; A61P 43/00

(52) U.S. Cl. 424/43

(57) ABSTRACT

Disclosed here is a stable, aqueous aerosol oil-in-water emulsion foam composition useful in dispensing a wide variety of therapeutic agents as an alternative to solid or liquid dosage forms which may be difficult to swallow or where the quantity of liquid intake is to be limited. More particularly, this invention discloses foam compositions containing a calcium compound in high concentration to be used as food supplement with an unexpectedly pleasant taste and little after-taste allowing better patient compliance.

Tablets and capsules are the most common dosage forms for the oral administration of nutritional, medicinal, or other therapeutic products. It is well-known that these dosage forms are unacceptable for use by patients who may have difficulty swallowing tablets and capsules, particularly when the size of dosage form is large or where the taste of the dosage form is offensive. All of these disadvantages result in frequent lack of patient compliance.

The common alternatives to conventional tablets and capsules are chewable tablets and aqueous or liquids such as syrups, suspensions and elixirs. Such dosage forms are commonly used for antacids, analgesics, cough and cold medications, antibiotics, vitamins and many other nutritional or medicinal products. In general, these forms do not significantly improve the taste of a medication or make it easier to swallow larger doses. For example, antacids in either chewable tablet or aqueous suspension form are generally disliked because they are gritty, astringent and leave an unpleasant aftertaste.

Although aerosol packaging has found high consumer acceptance in many areas, including pharmaceutical products such as inhalants, it has not heretofore been considered for use in formulations requiring a high concentration of suspended solids, i.e., greater than 5 to 13%, because a high solids content usually causes malfunctioning of the aerosol valve. It is neither economical nor practical to dispense therapeutic agents in the very dilute formulations which would be required for dispensing through an aerosol valve. Moreover, such dilute formulations usually produce an uncontrollable and immeasurable spray, thereby making it difficult to control or measure the amount of the formulation being dispensed. A further difficulty with aerosol packaging is that most aqueous aerosol solutions would be unacceptable for dispensing medications because the dissolution of the active ingredient prior to ingestion could produce an unpleasant taste.

There are several prior art patents which disclose anhydrous aerosol foams. For example, U.S. Pat. No. 3,770,648 discloses an anhydrous aerosol foam composition for external use which incorporates a silicone resin in a solution of organic solvents to produce a stable “quick breaking” foam when the foam is rubbed into or spread over a surface on which it has been deposited. The '648 patent does not disclose any anhydrous foam products which are suitable for ingestion. It also does not teach any type of dosable or meterable foam for dispensing high concentrations of solid therapeutic agents.

The U.S. Pat. No. 3,849,580 discloses an aerosol dispensing system which delivers non-aqueous butter-like edible fat compositions in a foam form. These foams contain no foaming agent and are intended to be used as food spreads.

The U.S. Pat. No. 4,425,164 teaches the preparation of an aerosol spray cookware lubricant composition similar to the product which is commercially available in food stores under the trademark “PAM.” This spray is formed from a mixture of a vegetable oil solution of an emulsifier (lecithin) in admixture with at least 10%, preferably 20 to 30%, of a hydrocarbon propellant and up to 15%, preferably 3 to 10%, of suspended flour or starch particles. The resulting product is a spray in which the particles serve as a visual indicator that the spray is being uniformly applied to the cooking surface. There is no disclosure or suggestion of a directly ingestible stable foam product capable of yielding repeatable, measurable doses of an active, solid therapeutic agent from an aerosol container.

Other disclosures of vegetable oil, lecithin-containing edible aerosols (U.S. Pat. Nos. 4,188,412 and 3,821,007) also indicate that such materials are sprays rather than foams. These patents additionally teach that foaming action would be undesirable in such a product.

The U.S. Pat. Nos. 4,439,367 and 4,752,465 teach anhydrous edible aerosol foams as means of administering drugs and food supplements which allow masking bad taste and sensation.

### DETAILS OF INVENTION

It has now been found that a stable, edible, oil-in-water emulsion aerosol foam or whip capable of containing a large quantity of dispersed solids and liquids can be prepared from a foamable, edible liquid oil, water and emulsifying agent; a foaming agent; water and controlled amounts of a food grade propellant which are sufficient to produce a stable foam rather than a spray. The foam, as delivered from an aerosol canister, has the consistency of whipped cream, is stable for extended periods and resists growth of micro-organisms so that refrigeration is generally not required. It can be safely ingested so that it is ideal as a carrier for medicines, vitamins, minerals or other solid therapeutic agents. Additionally, drugs that are not soluble in the ingredients of the foam can be kept out of solution in the mouth. Thus, the foam of this invention assists in masking the taste of bitter drugs such as acetaminophen, smoothing the taste of chalky astringent drugs such as magnesium hydroxide and aluminum hydroxide, and making it easier to administer large amounts of high dosage medications such as calcium supplements.

Through use of this invention, antacids in a whip or aerosol foam become extremely palatable and easy to swallow making it possible to prepare products containing a much larger amount of active antacid ingredients so that greater effectiveness and compliance can be achieved than is possible with conventional chewable antacid tablets or aqueous antacid suspensions. For example, a typical dose of magnesium hydroxide as a liquid laxative is 2 to 4 tablespoons, whereas the foam of the invention requires only 2 to 3 teaspoons to deliver the same amount of active ingredient. Similar desirable results can be achieved with antitussives such as dextromethorphan, antihistamines such as chlorpheniranime, decongestants such as pseudoephedrine and local anesthetics such as benzocaine or dyclonine.

The stability of the novel foam formulation enables it to be controlled allowing accurate measurements for dosing purpose such as by using a spoon or a similar device for oral administration, or measured into an applicator for rectal or vaginal administration. Obviously, such foam is capable of being packaged in small, portable aerosol containers (the size of a typical breath spray container) which may be easily transported as well as in shaving cream-sized containers for home use.

The stability of the whip or foam product reported also enables the present invention to be utilized as a base for food products. Thus, it can be combined with various sweetening and flavoring agents to provide a whipped cream-type food product which needs no refrigeration or preservatives. Sweetening and flavoring agents, of course, may also be employed to enhance the flavor of pharmaceutical products to further enhance the likelihood of patient compliance.

While not wishing to be limited to any particular theory, it is believed that the formulations of the present invention are capable of achieving the foregoing results without valve clogging due to a novel combination of ingredients that:
1. Produce a high viscosity formulation capable of keeping the small solids particles dispersed.
2. Lubricate the aerosol valve due to the presence of oil and surfactants.
3. Prevent the formation of hard aggregates (“caking”) during the shelf life due to the combined action of polymers and surfactants used in the invention.

The foamable, edible liquid oils utilized in the present invention are varied and of no critical significance. Typical among the edible organic oils useful for the present invention include soybean oil, partially hydrogenated soybean oil, linseed oil, corn oil, peanut oil, sunflower oil, cottonseed oil, olive oil, castor oil, liquid petrolatum, oleic acid, lauric acid, and mono- and diglyceride oils. As indicated above, the basic criteria for the selection of the liquid oil is that it be foamable and edible.

Typically, the edible oils utilized in the present invention are present in the formulation in a percentage of 1 to 80% by weight of the total composition. A preferred range is 2 to 40% by weight of the total composition. The amount of oil may be varied based upon the nature and amount of the other ingredients in the formulation, such as the amount of dispersed solids. Ordinarily, the percentage amount of each other ingredient in the formulation is first selected and the oil is the ingredient added to bring the formulation to 100% wherein the aqueous portion is not less than 5% to obtain an oil-in-water emulsion.

Foaming agents utilizable in the present invention are selected from the group consisting of lecithin and various polyol fatty acid esters and mixtures thereof. Lecithin is the commercial name for a class of naturally occurring compounds derived from soybeans and these compounds are phosphatides and phospholipids. The principal components of lecithin are a naturally occurring mixture of phosphatidyl choline, phosphatidyl ethanolamine, inositol phosphitides and related phosphorous containing lipids. Chemically, lecithin is described as phosphatidyl choline and is a mixture of the diglycerides of stearic, palmitic and oleic acids linked to the choline ester of phosphoric acid. It is available commercially as a 60% solution in soybean oil or as a granular powder essentially free of soybean oil. A hydroxylated lecithin, modified to increase the hydrophilic properties is also commercially available. This hydroxylated lecithin is commonly supplied as a 60% solution in soybean oil.

The polyol fatty acid esters utilizable in the present invention are commercial products and are comprised of three types: glycerol esters of fatty acids, polyglycerol esters of fatty acids and sorbitan esters of fatty acids. Additionally, a combination of any of the polyol fatty acid esters may be utilized in the present invention.

The polyol fatty acid esters allow the formation of a foamable, edible oil-in-water emulsion due to the enhanced consistency they provide to the formulation. This gives a much less oily feel in the mouth and releases the suspended medicament more rapidly in the stomach. This combination also causes the release of a suspended or dissolved medicament faster in the stomach as compared to an oily outer phase as disclosed by U.S. Pat. No. 4,752,465. As it is necessary for the final product to be edible, the polyol esters are approved for internal use by the corresponding regulatory authorities.

The foaming agent utilized in the present invention is present in an amount ranging from 0.5 to 40% by weight. The amount of foaming agent utilized depends upon the particular foaming agent, the particular foamable oil being utilized in the oil-in-water emulsion system and the propellant system. A preferred range of foaming agent is from about 1 to 10% by weight of the composition, with 1.3% being especially preferred. It is a particularly desirable additional feature of the foaming agents that they possess surfactant properties and, therefore, affect the rate at which the insoluble solid active ingredient of the foam is released in the body. Accordingly, some variations in the amount of foaming agent in a particular formulation may be purposely chosen based on the nature of the solid active ingredient in order to control the rate of release.

The edible propellant can be selected from the class of hydrocarbons that are gaseous under atmospheric pressures and liquefy when compressed, or certain edible fluoroalkanes such as Norflurane (HFA 134a), HFA 227ea, or nitrous oxide or other suitable propellants compatible with the disclosed formulation or mixtures thereof.

The use of hydrofluoroalkanes is advantageous due to their non-flammability, which results in reduced hazard for the consumer and for the manufacturing operation but there is a distinct cost disadvantage in using these propellants.

The most commonly used hydrocarbon propellants are propane, butane and isobutane. Propane is approved for use in ingested products and can be obtained commercially in an odorless and tasteless form which is ideally suited for use in preparing the whip of the present invention. Since these liquefied gases are soluble in the oil vehicle of the composition, there is a resulting reduction in their vapor pressure. Therefore, it is most advantageous to use propane since it has the highest pressure of the three hydrocarbon propellants and, even when dissolved in the low concentrations normally employed in this invention, produces a product with a pressure of 30-40 pounds per square inch over atmospheric pressure. This pressure is required to eject the foam from the container and produce stable, dense foam which can be measured onto a spoon to facilitate appropriate dosing prior to administration. Further, since propane is soluble in the oil base, there is very little pressure drop from the first to the last actuation of the aerosol valve and satisfactory foam is produced when each dose is expelled.

The amount of propellant used is critical to avoid generation of undesirable spray rather than the desired stable, measurable foam. The amount of propellant in the range of from 1-40% by weight is desirable and 3-11% by weight is the preferred concentration. The amount of propellant used may vary somewhat, depending upon the nature and amount of the other ingredients in the composition but, in all cases, the lowest amount sufficient to form a stable, measurable foam without forming a spray is selected.

Propellants other than the liquefied hydrocarbon gases can be used including compressed gases like nitrogen, nitrous oxide and carbon dioxide, but they do not produce the most desirable foams over the life of the product in use.

The edible oil-in-water emulsion aerosol foam of the present invention may be used as a vehicle to deliver a large variety of active pharmaceutical materials or cosmetic ingredients. Additionally, the foam itself can be used as a base for various sweetening and flavoring agents in order to dispense food items. The active pharmaceutical materials which can be incorporated in the foam of the present invention include can be any of the common antacids, analgesics, antitussives, laxatives, calcium supplements, vitamins, minerals, or any other type of therapeutic agent.

A particularly important and surprising feature of the foams of this invention is their ability to suspend high concentrations, i.e., up to 50% by weight, of solids and liquids, and mask their taste when ingeted. Thus, the need to mask the taste of bitter water soluble drugs such as acetaminophen, or smooth the taste of chalky astringent drugs such as magnesium hydroxide and aluminum hydroxide is markedly reduced. This greatly simplifies the formulation of such drugs and obviates any potential problems with absorption and side effects of additional excipients required for taste masking. Additionally, the foam can incorporate flavoring agents to further enhance its organoleptic properties.

The foam of the present invention can contain up to 50% by weight of suspended solid particles without any appreciable valve malfunctioning, and will usually contain in excess of 15% by weight of such solid particles since a primary purpose of the foam system is to deliver a high concentration of the active ingredient in the solid particles in a relatively small dose. This ability of the invention to suspend high percentage of solids, without valve malfunctioning, enables the aerosol foam system of the present invention to be utilized for a wide variety of formulations. The reasons for the unique ability of the foams to suspend such a high concentration of solids without valve clogging are not fully understood, but believed to result from a combination of the small particle size, the high viscosity of the foam formulation due to its low propellant content, all of which aid in keeping the particles dispersed with less agglomeration and settling while maintaining the lubricating effect of the oily phase and surfactants on the valve.

The foams of the present invention are prepared by conventional formulating techniques. Thus, typically, the foamable edible oil-in-water emulsion and the foaming agent are mixed together along with other soluble ingredients of the composition. The solid or liquid to be dispersed or dissolved is added to specific phases where it is to be dispersed or dissolved. The batch is then submitted for aerosol filling to an aerosol can. An aerosol valve is placed on the can and the can is crimped. The food grade propellant is then added by pressure filling.

In addition to the active solid to be dispersed or dissolved in the foam and the essential ingredients of the foam, there may also be incorporated in the foams of the present invention any of a variety of additives or a combination thereof, commonly added to aerosol compositions or to toiletries, cosmetics, or pharmaceuticals. Typically, such additives are those such as emollients, lubricants, humectants, abrasives, and perfumes.

It will be apparent to those skilled in the art that many modifications, both of materials and methods, may be practiced without departing from the purpose and intent of the disclosure.

Calcium is a mineral that is widely used as food supplements for several groups of population, such as post-menopausal women, renal failure patients, osteoporosis suspects and children in general or others where calcium deficiency is suspected. The calcium delivering preparations, as commercially available, have a relatively chalky taste and sensation when swallowed, which often makes them less palatable resulting in lack of compliance. Because of the need to administer relatively larger quantities of calcium with relatively low amounts of water in the chronic renal failure population, the present invention is particularly suitable for them.

The peculiar physical characteristics of calcium powder pose an enormous challenge to formulators to prepare a highly concentrated, relatively taste-free formulation. However, contrary to the teachings of the abovementioned patents, the present invention relates to water-containing foam with a certain amount of fatty components and surfactants that produce an oil-in-water emulsion, which eliminates the direct contact of calcium powder with the taste buds of users and thus eliminating the objectionable taste and feeling in the mouth. The formulations developed in this invention allow a large quantity of water to be incorporated in the preparation such as in excess of 30% by weight without adversely affecting the taste-masking ability of the composition. Furthermore, the possibility of including water also allows incorporation of water soluble sweetening agents which improve the taste-masking properties of the formulation.

An additional advantage of the inclusion of water in the present invention is that it eliminates the need to disperse solids in oily phase to mask their taste; as a result, no dispersed solid is required to avoid oily or fatty unpleasant taste, because the preparation described herein is an oil-in-water emulsion as edible cream. Therefore, it provides a pleasant creamy sensation without the need of high amounts of dispersed solids as required by the previous teaches as can be read on lines 33 to 40 of U.S. Pat. No. 4,752,465.

Additionally and contrary to the teachings of U.S. Pat. No. 4,439,367 on lines 35-39, a non-hydrocarbon propellant, namely HFA 134a can be used. The HFA 227ea can also be used for this preparation without deteriorating its stability. The preparation is therefore non-flammable, which provides additional safety in its manufacture, use and storage.

We have tested calcium carbonate to have a worst case evaluation of the formulation because the calcium preparations are generally unpleasant to taste. However, the formulation with some variations could also be used for other calcium compounds, such as calcium acetate, calcium phosphate, calcium citrate, calcium tartrate, etc.

Aerosol foams consist of, at least, a biphasic system having a gaseous phase dispersed in a viscous liquid or solid. The invented foam formulations consist of viscous liquids containing:
1. Fatty components such as animal fats or oils either hydrogenated or not, vegetable oils hydrogenated or not
2. Sweetening agents, such as saccharin, saccharin salts, cyclamate salts, sucrose or other sugars, polyols such as sorbitol, xylitol, glycerin or the like.
3. Water as continuous phase of the foam where sweeteners are dissolved and fatty components emulsified.
4. Emulsifying agents to get stable emulsion of fatty components in the aqueous continuous phase, such as polysorbates, sorbitan esters, sugar esters or other suitable edible surfactants.
5. Flavoring agents to give a certain distinctive taste to the foam
6. Propellant

The preparation must be packaged into a pressurized container and a dispensing valve should be crimped onto it so that when the valve is actuated foam of propellant bubbles dispersed in the aqueous emulsion of fatty components is immediately produced for direct consumption. All ingredients should be food or medicinal grade.

### EXAMPLE 1

| Component | Amount/100 G | Function |
|---|---|---|
| Calcium Carbonate | 20.82 | Active ingredient |
| Xantham Gum | 0.60 | Viscosity-increasing agent |
| Glyceryl monostearate | 0.30 | Foam and Emulsion stabilizer |
| Polysorbate 80 | 0.74 | Foam and Emulsion stabilizer |
| Propylene Glycol | 0.25 | Foam and Emulsion stabilizer |
| Monostearate | | |
| Hydrogenated edible oil | 3.64 | Oil phase |
| Sunflower oil | 4.54 | Oil phase |
| Sorbitol 70% | 17.82 | Sweetener |
| Xylitol | 6.36 | Sweetener |
| Butylated | 0.019 | Antioxidant |
| hydroxytoluene | | |
| (BHT) | | |
| Vanilla flavor | 0.091 | Flavor |
| Water | 35.72 | Aqueous external |
| | | phase of emulsion |
| Propellant (HFA 134a) | 9.10 | Propellant |

Fatty components (glyceryl monostearate, hydrogenated edible oil and sunflower oil) and butylated hydroxytoluene are melted and dispersed into water containing the dissolved xantham gum and polysorbate 80. Sweeteners (sorbitol 70% and xylitol) and vanilla flavor are added to the previously obtained emulsion. The concentrate is filled into aerosol cans. A valve is crimped onto the can and the propellant (HFA 134a) is filled through the valve. Units are placed into a water bath at 40° C. to detect defective crimping.

### EXAMPLE 2

| | Amount/ | |
|---|---|---|
| Component | 100 G | Function |
| Calcium carbonate | 20.82 | Active ingredient |
| Hypromellose | 0.60 | Viscosity increasing agent |
| Glyceryl monostearate | 0.45 | Foam and Emulsion stabilizer |
| Polysorbate 80 | 0.74 | Foam and Emulsion stabilizer |
| Lecithin | 0.10 | Foam and Emulsion stabilizer |
| Hydrogenated edible oil | 3.64 | Oil phase |
| Sunflower oil | 4.54 | Oil phase |
| Sorbitol 70% | 17.82 | Sweetener |
| Xylitol | 6.36 | Sweetener |
| Butylated hydroxytoluene | 0.019 | Antioxidant |
| (BHT) | | |
| Orange flavor | 0.181 | Flavor |
| Water | 35.63 | Aqueous external phase of |
| | | emulsion |
| Propellant (HFA 134a) | 9.10 | Propellant |

Fatty components (glyceryl monostearate, hydrogenated edible oil and sunflower oil) and butylated hydroxytoluene are melted and dispersed into water containing the dissolved hypromellose, lecithin and polysorbate 80. Sweeteners (sorbitol 70% and xylitol) and orange flavor are added to the previously obtained emulsion. The concentrate is filled into aerosol cans. A valve is crimped onto the can and the propellant (HFA 134a) is filled through the valve. Units are placed into a water bath at 40° C. to detect defective crimping.

Interestingly none out of 10 units actuated until exhaustion exhibit any malfunction or valve clogging. This is fairly remarkable if it is taken into account that the formulation contains approximately 20% of solids suspended in it.

What is claimed is:

1. An edible, aqueous aerosol foam composition comprising an oil-in-water emulsion, a foam stabilizer/emulsifier, a propellant and optionally a viscosity-enhancing agent.

2. The composition of claim 1 wherein said composition additionally contains a therapeutic agent suitable for administration to humans and animals in a suitable therapeutic dose.

3. The composition of claim 2 wherein the therapeutic agent consists of a calcium compound.

5. The composition of claim 1 wherein said composition is packaged in a pressurized container fitted with an aerosol valve.

6. The composition of claim 1 wherein said propellant being present in an amount sufficient to produce a stable, measurable foam but insufficient to produce a spray when said composition is ejected through said aerosol valve.

7. The composition of claim 1 wherein said propellant comprises 1 to 40% (w/w) of said composition.

8. The composition of claim 1 wherein said propellant is a hydrocarbon.

9. The composition of claim 1 wherein said propellant is hydrofluorocarbon HFA 134a or HFA 227ea or a mixture thereof.

10. The composition of claim 1 wherein said propellant is nitrous oxide.

11. The composition of claim 1 wherein said oil in the oil-in-water emulsion is selected from the group consisting of soybean oil, partially hydrogenated soybean oil, linseed oil, corn oil, coconut oil, peanut oil, sunflower oil, cottonseed oil, olive oil, castor oil, liquid petrolatum, oleic acid, lauric acid and mono- and diglyceride oils, and hydrogenated edible oil.

12. The composition of claim 1 wherein said water proportion in the oil-in-water emulsion comprises at least 5% (w/w) of said composition.

13. The composition of claim 1 wherein said foam stabilizer/emulsifier is selected from the group consisting of lecithin, propylene glycol esters, glycerol esters of fatty acids, sorbitan esters of fatty acids, polyethylene glycol esters of fatty acids, sugar esters of fatty acids, oleic acid and mixtures thereof.

14. The composition of claim 1 wherein said foam stabilizer/emulsifier comprises 0.2 to 20% (w/w) of said composition.

15. The composition of claim 1 wherein said viscosity-enhancing agent is selected from a group consisting of gelatin, cellulose derivatives, agar-agar, guar gum, xanthan gum, alginates, povidone and mixtures thereof.
